(19) United States
(12) Patent Application Publication
(10) Pub. No.: US 2022/0330932 A1
(43) Pub. Date: Oct. 20, 2022

(54) SUTURING DEVICE COMPRISING DIES

(71) Applicant: Arkady Veniaminovich DUBROVSKY, Moscow (RU)

(72) Inventor: Arkady Veniaminovich DUBROVSKY, Moscow (RU)

(21) Appl. No.: 17/640,867

(22) PCT Filed: Aug. 19, 2020
(86) PCT No.: PCT/RU2020/000442

§ 371 Date: Mar. 7, 2022

(30) Foreign Application Priority Data
Sep. 5, 2019 (RU) 2019128022

(51) Int. Cl. A61B 17/00; A61B 17/28

(52) U.S. Cl. CPC A61B 17/00491; A61B 17/282

(57) ABSTRACT

A suturing device comprises a housing (1) having operating handles at one end and two clamping working jaws (2 and 3) at the other end, at least one of which (jaw 3) is mounted for movement relative to the second. The device also comprises: a receptacle (capsules 18) having a quick-setting, biocompatible, fluid material, which is used as a means for fastening tissues, wherein at the outlet of said receptacle is a controllable screen, for example a membrane, which is destroyed when the pressure in the receptacle is increased to a set level; and a mechanism for feeding the fluid material into a suturing zone. The working jaw (3) is hollow, and disposed therein are capsules (18) which comprise the fluid material and communicate, via the outlet, with dies (21) which are designed in a wall of the working jaw (3) that interacts with the suturable tissues. The mechanism for feeding the fluid material is designed to be capable of increasing the pressure inside the capsules (18) and forming streams of the fluid material which pass through the dies (21) at a pressure sufficient for passing through the suturable tissues.

### TECHNICAL FIELD OF THE INVENTION

The invention relates to medical technology and more specifically relates to a suturing device intended for suturing biological tissues, but can also be used in other fields.

### BACKGROUND OF THE INVENTION

The existing surgical suturing devices mainly use metal agraffes made of 40 KNHM alloy or titanium. The agraffes are inert and biocompatible with human tissues. However, they are permanent foreign matters in the human body, with scar tissues developing therearound. The agraffes can change their position during examination on nuclear magnetic resonance (NMR) devices, which is very dangerous.

The prior art discloses Covidien and Ethicon suturing devices using agraffes made of polylactides (TA Premium, Poly GIA™ 75, Roticulator 55 Poly™). These agraffes dissolve after a certain amount of time. They are larger than metal ones and are used only in gynecology and urology.

A number of patents for suturing devices describe the possibility of using the existing synthetic absorbable stitches on atraumatic needles or absorbable fasteners (patents RU 2076639 dated 10 Jan. 1993, RU 2074651 dated 14 Sep. 1993, RU 2119771 dated 7 Oct. 1993, RU 2120240 dated 5 Mar. 1996, RU 2310405 dated 13 Jan. 2004, RU 2328228 dated 11 Apr. 2007, RU 2381756 dated 18 Jun. 2008, RU 146042 dated 5 May 2014, RU 159052 dated 6 May 2015, RU 184491 dated 15 May 2018). These devices are more complicated than suturing devices-staplers, since additional mechanisms are required to fix the ends of the stitches or fastening elements.

At present, two-component and multicomponent compositions have been developed, which instantly cure in air or under the influence of appropriate radiation.

Similar materials have been obtained by a number of researchers. They are reported, for example, by Spiber (Japan), AMSilk (Germany), Nexia Biotechnologies (Canada), Bolt Treads (USA), etc.

Application RU 2019105155, dated 25 Feb. 2019 and entitled “Suturing device”, describes various embodiments of surgical suturing devices and surgical forceps using biocompatible fast-curing materials for connecting (suturing) biological tissues. In the devices and forceps described in the above-indicated application, the fast-curing mass is introduced into the tissues to be sutured through the channels of hollow needles after the tissues are pierced with the needles during the removal of the needles from the tissues. The complexity of these devices is due to the presence of the hollow needles and a mechanism for advancing the needles into the tissues and withdrawing them from the tissues.

### SUMMARY OF THE INVENTION

It is an objective of the invention to create a design of a suturing device suitable for connecting biological tissues by using fast-curing fluid masses, including for suturing lung and liver tissues with the least possible trauma for such tissues.

The objective above is achieved by a surgical suturing device comprising a housing having control handles at one end and two clamping working jaws at another end, at least one of which is arranged to move relative to the other. The device further comprises: a receptacle having a biocompatible fast-curing fluid mass used as a means for fastening tissues, with a controllable damper being mounted at the outlet of the receptacle; and a mechanism for feeding the fluid mass into a suturing zone. According to the invention, one of the working jaws is made hollow and has the fluid mass receptacle arranged therein and having an outlet connected to dies that are provided in the wall of the working jaw interacting with the tissues to be sutured. The mechanism for feeding the fluid mass is configured to provide a pressure increase inside the receptacle and form jets of the fluid mass which pass through the dies under a pressure sufficient for passing through the tissues to be sutured.

It is desirable to select the diameter of the dies based on the thickness and density of the tissues to be sutured. In this case, the diameter of the dies may vary from a few fractions of millimeter downwards. The fluid mass squeezed out through the dies penetrates the tissues to be sutured and, quickly curing in the tissues, forms stitches that connect the tissues. However, to obtain a tight suture (for example, a lung tissue suture), it is desirable that the stitch is arranged not only inside the tissues, but also on the surface of the tissues, thereby forming a closed ring.

To achieve this, it is possible to form grooves on the surfaces of the working jaws interacting with the tissues to be sutured. The grooves are configured to be filled with the fluid mass during the suturing process and form closed annular fasteners that do not have knots which are usually present, for example, in a handmade suture, which leads to an increase in trauma.

It is also possible to arrange thin patches, which is made of a biocompatible fast-absorbable material capable of interacting with the fluid mass during the suturing process and forming a tight suture, on the surfaces of the working jaws interacting with the tissues to be sutured.

The mechanism for feeding the fluid mass may be connected to an external pressure source.

A damper, which automatically opens when the pressure in the receptacle increases to a predefined value, is arranged between the receptacle with the fast-curing fluid mass and the dies.

In a preferred embodiment, the fluid mass receptacle is made as a number of discrete capsules that are arranged in the cavity of the jaw above the dies and connected to the dies. In this case, each capsule has an outlet protected by a membrane which is destructible in response to a pressure increase in the capsule or when interacting with sharp edges of the dies in response to the pressure increase in the capsule.

In another embodiment of the device, the fluid mass may be arranged in the single receptacle arranged in the cavity along its entire length and connected to the dies by the damper made as a number of rotary valves mounted on a single axis. In this case, an actuator for changing a position of the valves is synchronized with a mechanism for changing the pressure in the capsule.

In yet another embodiment, the fluid mass receptacle may be connected to the dies by the damper that is formed by three plates having matching holes. Out of the plates, a middle plate is movable relative to the other plates and, when shifted, opens and closes the holes. In this case, an actuator for changing a position of the middle movable plate is synchronized with the mechanism for changing the pressure in the receptacle.

In a preferred embodiment, the dies are arranged in one row or in two or more rows in a checkerboard pattern.

The technical result achieved by using the invention is that the dies may be made significantly smaller in diameter compared to hollow needles. At the same time, the tissue trauma caused by the dies having a small diameter and the stitches accordingly formed by them is comparable to the trauma caused to tissues by a piercing pointed needle of the same diameter.

To connect cartilage or bone tissues, it is necessary to form channels pre-filled with a connecting substance. The channels may be formed by using laser beams or drills of an appropriate diameter.

### BRIEF DESCRIPTION OF THE DRAWINGS

The invention is further explained by a description of certain embodiments thereof and by the accompanying drawings in which:

FIG. 1 shows a general side view of one of suturing device types, with a cutaway near a housing and a fixed jaw.

FIG. 2 shows a magnified cutaway view of a working fixed jaw.

FIG. 3 shows a general side view of an endoscopic forceps having a pressing-through device, with a cutaway near a housing and a fixed jaw.

FIG. 4 shows a general side view of a surgical forceps having a pressing-through device, with a cutaway near one of working jaws.

FIG. 5 shows a general side view of a suturing device having parallel jaws, with a cutaway near a housing and a movable jaw.

FIG. 5a shows a magnified cutaway view of the movable jaw of the device shown in FIG. 5. A damper is made as a movable plate, the damper is closed.

FIG. 5b shows a magnified cutaway view of the movable jaw of the device shown in FIG. 5. The damper is made as valves on a single axis, the damper is closed.

FIG. 5c shows the same as FIG. 5b, the damper is open.

FIG. 6 shows the same as FIG. 5, the embodiment with a receptacle made as a number of capsules.

### DETAILED DESCRIPTION OF THE INVENTION

The embodiments of suturing devices according to the invention, which are shown in the figures, include the following elements: 1—a device housing, 2—a fixed working jaw; 3—a movable working jaw; 4—an axis of the movable jaw; 5—a handle of the movable jaw; 6—an axis of the handle of the movable jaw; 7—a return spring of the handle of the movable jaw; 8—a rod finger of the movable jaw; 9—a rod of the movable jaw; 10—an axis of the rod of the movable jaw; 11—an area with teeth at the proximal end of the rod of the movable jaw; 12—a lock-hook for the teeth of the rod of the movable jaw; 13—a spring for the hook 12; 14—a screw handle for pressing through a fast-curing mass; 15—a hinge between the handle and the rod; 16—a rod for pressing through the fast-curing mass; 17—a pusher for pressing through the fast-curing mass; 18—capsules with the fast-curing mass; 19—a damper; 20—a rod of the damper; 21—dies; 22—a jaw clamp handle with a rod; 23—a working jaw with a rod; 24—a second forceps handle; 25—a second working jaw; 26—a forceps axis; 27—an absorbable biocompatible patch on the working jaws.

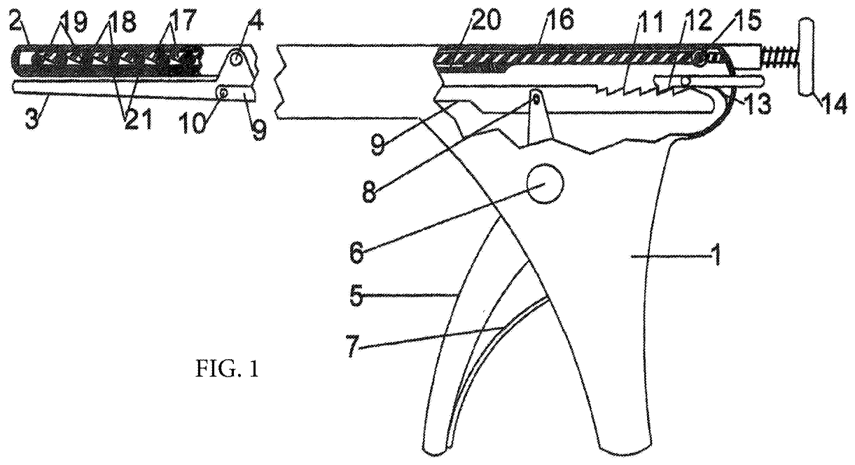

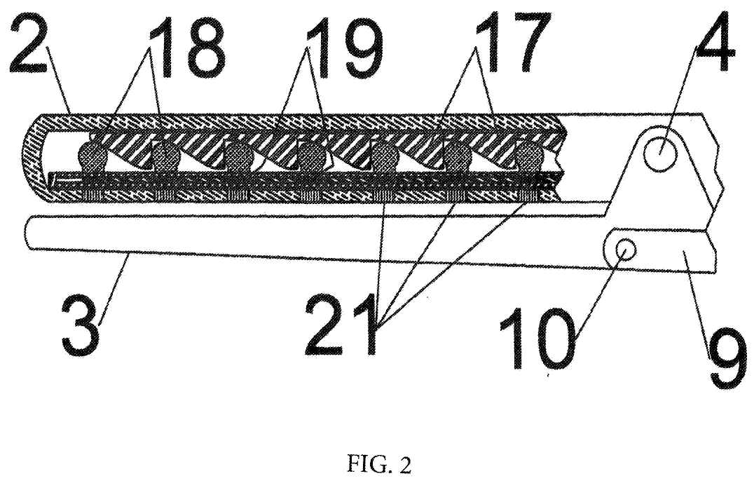

The suturing device shown generally in FIGS. 1 and 2 comprises a housing 1 having a fixed jaw 2 and a movable jaw 3. The movable jaw 3 is mounted on an axis 4 and is configured rotate around it. The movable jaw is controlled by using a handle 5 which is mounted on an axis 6 in the device housing 1. The handle 5 has a return spring 7. The short arm of the handle 5 has an oval hole through which a finger 8 of a rod 9 passes. The distal end of the rod 9 is connected to the movable jaw 3 through a hinge 10. At the proximal end of the rod 9, there is an area 11 with teeth that engage with a hook-lock 12 pressed by a spring 13 to the teeth 11. Thus, the hook-lock 12 fixes the movable jaw 3 in a predefined position. When raised, the hook-lock 12 unlocks the movable jaw.

A second handle 14 has a screw thread matching a female thread in the top part of the device housing 1. This allows the handle 14 to linearly move in the device housing 1 during rotation. The second end of the screw handle 14 is extended as a solid sphere rotating in a socket 15 at the proximal end of a rod 16, thereby forming a hinge. This hinge allows the rotary motion of the handle 14 to converted to the rectilinear movement of the rod 16.

As shown in FIGS. 1 and 2, the distal end of the rod 16 has a figured shape and functions as a pusher 17 for squeezing a fast-curing mass out of each capsule, i.e., it is substantially a final link of a mechanism for feeding the fluid mass into a suturing zone.

In some embodiments of the suturing device according to the invention, the mechanism for feeding the fluid mass into the suturing zone may be connected to an external pressure source (not shown in the figures).

FIG. 2 shows a magnified view of the working jaws of the device, with the working jaw 2 being shown in section. The working jaw 2 is made hollow and has a fluid mass receptacle. In this case, the fluid mass receptacle is made as individual capsules 18 distributed along the whole cavity of the jaw. The configuration of the rod 16 allows the entire content to be squeeze out of all the capsules 18 in the jaw 2. The bottom of each capsule is closed by a damper 19. The dampers may be made as membranes rupturing in response to a pressure increase in the capsule. However, there may be a common damper made, for example, as three parallel plates, out of which an internal plate is movable relative to the external ones. The plates have holes that are aligned in a certain position of the internal plate. The internal plate is connected to a rod 20 which, in turn, is connected to the rod 16. The movement of the rods 16 and 20 allows opening all the holes in the damper 19 synchronously with a pressure increase in the capsules 18. The capsules 18 have outlets connected by the dampers 19 to dies 21 provided in the wall of the working jaw 2 which is adjacent to the tissues to be sutured.

Thus, the rotation of the handle 14 functioning as a pressure changing mechanism provides a pressure increase in the capsules 18 and the opening of the dampers 19, thereby allowing the fast-curing mass to flow from the capsules 18 under a pressure through the dies 21. In this case, the fluid mass is fed through the dies 21 under a pressure sufficient to form thin jets which have a diameter similar to that of the dies 18 and penetrate the tissues to be sutured. The dies 21 may be arranged in one row or in two or more rows in a checkerboard pattern, taking into account the parameters of the dues and the surface size of the jaw adjacent to the tissues to be sutured. The dies 21 may have a different diameter depending on the thickness and density of the tissues to be sutured.

The adjacent holes of the dies on the outer side of the jaw 2 are connected by grooves. Similar grooves are present on the surface of the jaw 3 adjacent to the tissues to be sutured. This creates prerequisites for the formation of a closed stitch ring.

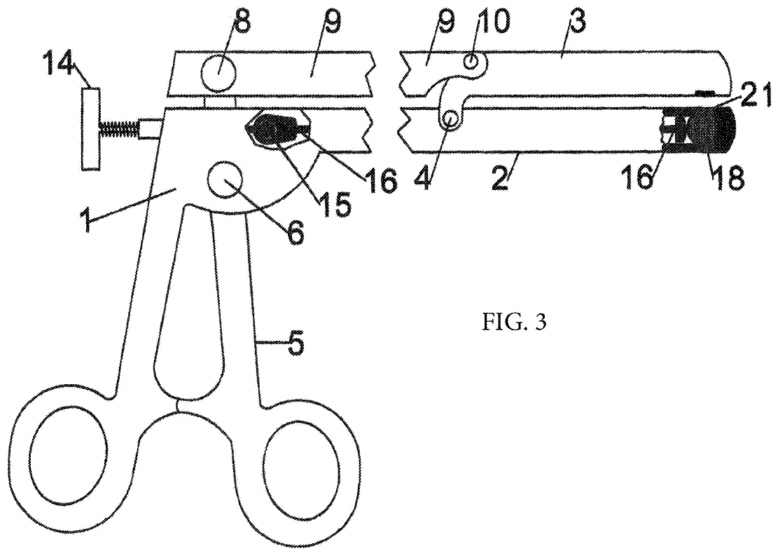

FIG. 3 shows a surgical endoscopic forceps. This forceps has an elongated housing 1 with handles arranged substantially perpendicular to a long housing axis. One of the handles is fixed. The housing ends with a fixed working jaw 2 in its distal part. A second working jaw 3 is movable relative to the fixed jaw 2 and is mounted on an axis 4. The jaw 3 is controlled by a movable handle 5 rotating around an axis 6. The small arm of the handle 5 is connected to a rod 9 by means of a finger 8. The distal end of the rod 9 is connected to the movable jaw 3 by means of a hinge 10.

The fixed and movable handles end with rings for surgeon's fingers and have ratchets by which it is possible to fix the position of the movable handle 5 and the movable jaw 3.

The housing also has a screw handle 14 that controls a rod 16. A hinge 15 is installed between the handle 14 and the rod 16 to convert the translation and rotational motion of the handle 14 to the rectilinear motion of the rod 16. The rod 16 is arranged in the housing 1 and the fixed jaw 2. The distal end of the rod 16 adjoins a fluid mass receptacle made, in this example, as a single capsule 18 arranged along the entire length of the cavity in the distal end of the fixed jaw 2. The capsule 18 is directly connected to dies 21 arranged in the wall of the fixed jaw 2 adjacent to tissues to be connected. A part of the capsule 18, at the junctions with the dies, is made as a membrane which ruptures in response to a pressure increase in the capsule, i.e., functions as a damper.

After the tissues to be connected are compressed with the forceps working jaws, the handle 14 is rotated, which, by means of the rod 16, ensures that the fast-curing mass is squeezed under a pressure out of the capsule 18 through the dies into the adjacent tissues to be connected, thereby forming thin jets having a diameter similar to that of the dies and penetrating the tissues to be sutured.

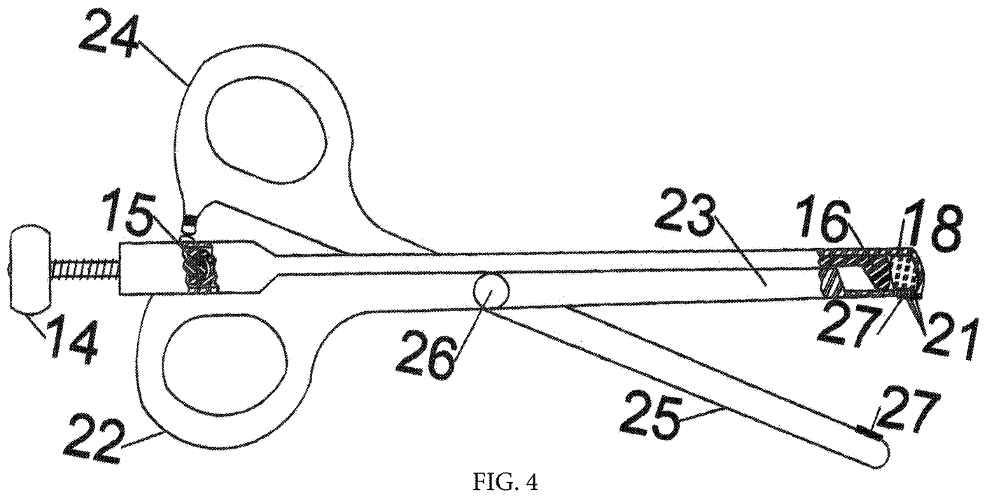

FIG. 4 shows a standard surgical forceps formed by two mirror parts having handles 22 and 24 with ratchets for surgeon's fingers and two working jaws 23 and 25. Both parts are connected by an axis 26.

There is a cavity at the distal end of one of the forceps jaws (the jaw 23 in this embodiment), in which the capsule 18 containing a fast-curing mass may be inserted. The lower surface of the capsule 18 is a membrane functioning as a damper—it ruptures in response to a pressure increase in the capsule or is pierced by sharp upper edges of the dies 18 when the capsule bottom displaces in response to the pressure increase in the capsule. The damper fits snugly against the dies 21 provided in the wall of the working jaw 23 adjacent to the tissues to be sutured.

The distal end of the rod 16 arranged along the working jaw 23 in a protecting casing adjoins the capsule 18 sideways. The rod 16 is connected by the hinge 15 to the screw handle 14 at its proximal end.

The distal ends of the jaw 23 (near the dies) and the opposite jaw 25 are provided with patches 27 which is made of biocompatible material dissolving after a certain amount of time and interacting with the jets of the fluid mass to form an annular tight suture during the suturing process.

After the tissues to be connected are compressed with the working jaws 23 and 25, the handle 14 is rotated, whereupon the rod 16 squeezes the fast-curing mass out of the capsule 18 through the dies into adjacent tissues to be connected.

Similar forceps will be widely used for hemostasis in any surgeries.

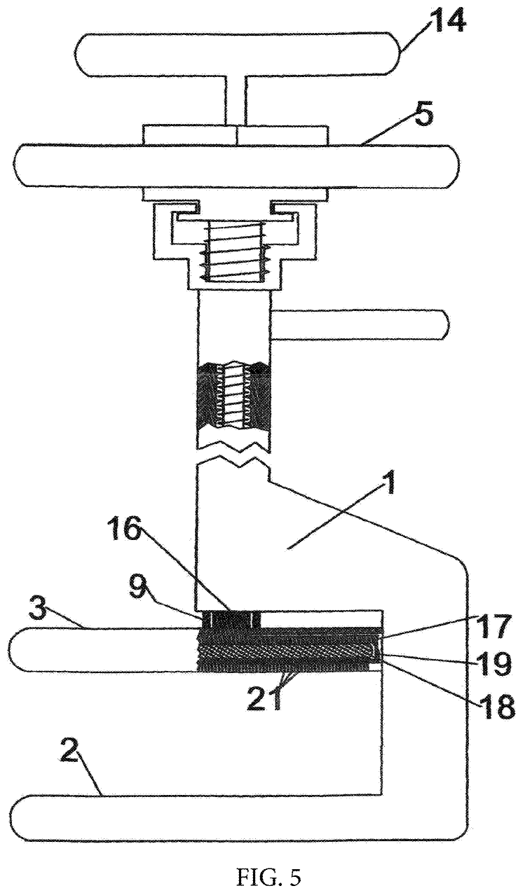

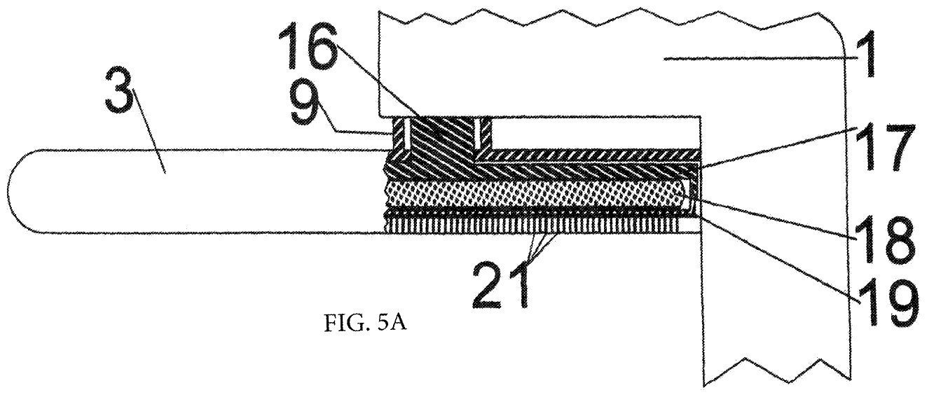

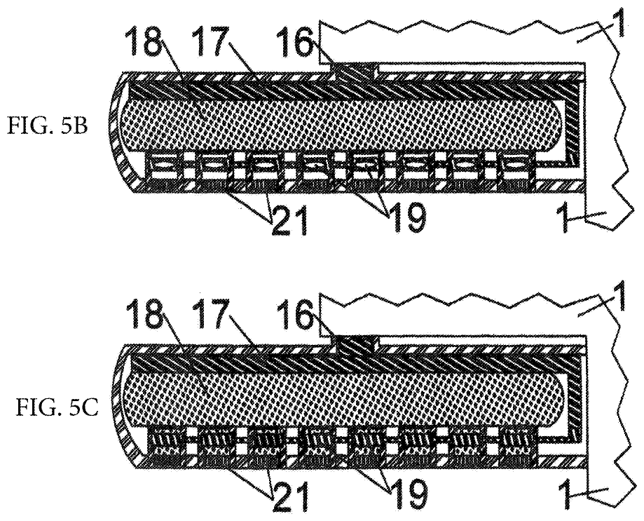

FIG. 5 shows a suturing device of UKL type, which is widely used in surgical practice. The device has an elongated housing 1 which ends with a fixed working jaw 2 arranged perpendicular to a longitudinal housing axis. A movable hollow jaw 3 is arranged parallel to the fixed jaw. A handle 5 of the movable jaw 3 is arranged in the proximal part of the housing 1 and is connected (by means of threaded connection) to the movable jaw 3 with the aid of a hollow rod 9 arranged inside the housing 1. The rotation of the handle 5 causes the movable jaw 3 to move to either side.

A screw handle 14 moving a rod 16 mounted in the hollow rod 9 is arranged above the handle 5. The rod 16 is connected to a pusher 17 which, in this embodiment, shaped as a plate arranged in the movable jaw 3 above a fast-curing mass receptacle (single capsule 18). One edge of the plate-pusher 17 protrudes beyond the edges of the receptacle and is beveled. It is more clearly visible in FIG. 5a showing a slightly magnified working jaw 3 of the device. The lower surface of the receptacle 18 fits snugly against the top surface of a damper 19 consisting of three plates and is connected, via the damper 19, to dies 21 provided in the bottom wall of the movable jaw adjacent to the tissues to be sutured. The lower plate of the damper 19 fits snugly against the dies 21. The middle plate of the damper 19 is movable and has holes that are similar to those made in the upper and lower fixed plates. When the middle plate is shifted to the right, and its edge protrudes beyond the edge of the capsule 18 and adjoins to the beveled edge of the pusher 17, as shown in FIG. 5a, the holes of the damper are misaligned—the damper is closed. When the pusher-plate 17 lowers down and begins to exert pressure on the capsule 18, its beveled edge of the pusher 17 displaces the middle plate of the damper 19 to the left, and the holes in the plates of the damper are aligned—the damper is open. Thus, the change in the position of the middle plate of the damper 19 is synchronized with the change in pressure in the fluid mass receptacle under the influence of the pressure changing mechanism—the pusher 17.

FIGS. 5b and 5c show another embodiment of the damper 19, in which the capsule 18 containing the fast-curing mass is connected to the dies 21 by channels which are provided with rotary valves arranged on a single axis. The axis is rotated by the beveled edge of the plate-pusher 17 when the plate-pusher starts pressing through the capsule 18. FIG. 5b shows holes in the channels, as the holes are provided perpendicular to the axis of the channels—all the valves are closed. In FIG. 5c, the valves are turned, i.e., open, and their holes are not visible.

With the aid the handle 5, the working jaws 2 and 3 connect and slightly squeeze the tissues to be sutured. Then, the handle 14 is rotated. The pressure from the handle 14 is transmitted to the capsule 18 through the rod 16 and the pusher 17, while the beveled edge of the pusher 17 shifts the middle movable part of the damper 19 (FIG. 5a) or rotates its axis (FIG. 5c). This provides the communication of the capsule 18 with the dies 21; once a predefined pressure value is reached, the fast-curing fluid mass from the capsule 18 flows through the dies 21 into the tissues to be sutured. A few seconds later, after the formed thin stitches are polymerized, the working jaws are moved apart, and the suturing device is removed.

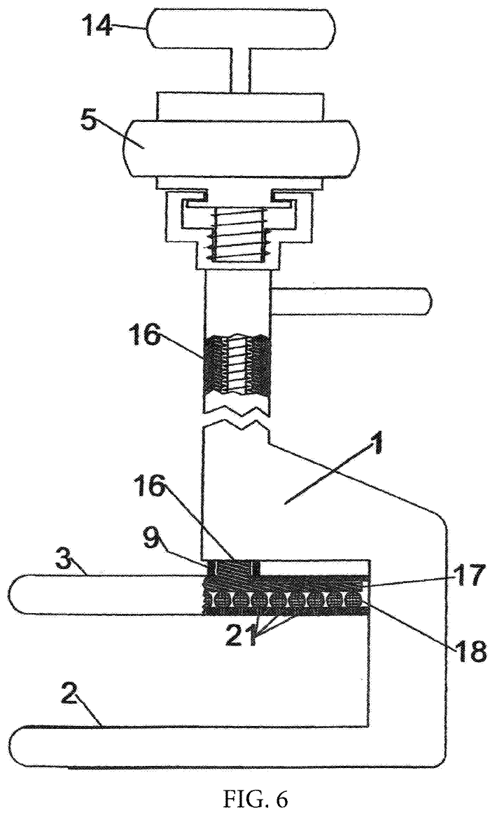

FIG. 6 shows the same suturing device as in FIG. 5, but instead of the common capsule 18, discrete smaller capsules 18 are used, which are arranged in one row or in two or more rows in a checkerboard pattern along the entire surface of the movable jaw adjacent to the tissues to be sutured. In this embodiment, the bottom of each capsule closely adjacent to the dies is made as a membrane that ruptures in response to a pressure increase in it. This embodiment is more economical in terms of fast-curing mass consumption and allows one to use ready-made stacks with capsules of various size and shape with appropriate dies, depending on the properties of the tissues to be sutured.

Thus, the proposed technical solution is intended for connecting biological tissues by forcing a biocompatible fast-curing fluid mass into tissues through dies. Embodiments of this technical solution may be used in combination with various surgical suturing devices and forceps. The configuration and diameter of the dies depends on the nature of the tissue to be sutured. This creates favorable conditions for tissue healing, speeds up the course of surgical interventions, solves the issues of final hemostasis without tissue coagulation, and will help automate a part of the surgical work in the future.

1. A suturing device comprising:
a housing having control handles at one end and two clamping working jaws at another end, at least one of the jaws being arranged to move relative to another of the jaws;
a receptacle containing a biocompatible fast-curing fluid mass as a means for fastening the tissues, the receptacle having a controllable damper mounted at an outlet of the receptacle; and
a mechanism for feeding the fluid mass into a suturing zone;
wherein one of the working jaws is made hollow and has the receptacle arranged therein, the outlet of the receptacle being connected to dies that are provided in a wall of the working jaw interacting with the tissues to be sutured;
wherein the mechanism for feeding the fluid mass is configured to provide a pressure increase inside the receptacle and form jets of the fluid mass which pass through the dies under a pressure sufficient for passing through the tissues to be sutured.

2. The suturing device of claim 1, wherein the dies have a diameter selected based on a thickness and density of the tissues to be sutured.

3. The suturing device of claim 2, wherein channels configured to be filled with the fluid mass during a suturing process and form closed annular fastening elements are provided on surfaces of the working jaws interacting with the tissues to be sutured.

4. The suturing device of claim 2, wherein patches made of biocompatible absorbable material capable of interacting with the fluid mass and forming a tight suture during a suturing process are arranged on surfaces of the working jaws interacting with the tissues to be sutured.

5. The suturing device of claim 2, wherein the mechanism for feeding the fluid mass is connected to an external pressure source.

6. The suturing device of claim 2, wherein the receptacle is made as a number of discrete capsules arranged in a cavity of the jaw above the dies and connected to the dies, and wherein each of the capsules has an outlet provided with a membrane that is destructible in response to the pressure increase in the capsule or when interacting with sharp edges of the dies in response to the pressure increase in the capsule.

7. The suturing device of claim 2, wherein the receptacle is arranged in a cavity of the jaw along the entire length of the cavity and connected to the dies via dampers made as rotary valves arranged on a single axis, and wherein an actuator for changing a position of the valves is synchronized with a mechanism for changing the pressure in the capsule.

8. The suturing device of claim 2, wherein the receptacle is connected to the dies by a damper consisting of three plates having matching holes, and wherein a middle plate of the three plates is movable relative to the other plates and, when moving, opens and closes the holes, and an actuator for changing a position of the middle movable plate is synchronized with a mechanism for changing the pressure in the receptacle.

9. The suturing device of any one of the preceding claims, wherein the dies are arranged in one row or in two or more rows in a checkerboard pattern.
